(19) United States
(12) Patent Application Publication
(10) Pub. No.: US 2007/0224620 A1
(43) Pub. Date: Sep. 27, 2007

(54) Compositions and methods for capturing and analyzing cross-linked biomolecules

(75) Inventors: Danette Hartzell, Madison, WI (US); Marjeta Urh, Madison, WI (US); Keith V. Wood, Mt. Horeb, WI (US)

Correspondence Address: SCHWEGMAN, LUNDBERG, WOESSNER & KLUTH, P.A., P.O. BOX 2938, MINNEAPOLIS, MN, 55402, US

(73) Assignee: PROMEGA CORPORATION

(21) Appl. No.: 11/704,150

(22) Filed: Feb. 8, 2007

Related U.S. Application Data
(60) Provisional application No. 60/771,558, filed on Feb. 8, 2006.

(51) Int. Cl. C12Q 1/68; G01N 33/53; C12Q 1/26

(52) U.S. Cl. 435/6; 435/7.1; 435/25

(57) ABSTRACT

Methods, compositions and kits to capture cross-linked protein complexes to a support matrix in a stable, covalent bridge of attachment are provided.

### CROSS-REFERENCE TO RELATED APPLICATIONS

This application claims benefit under 35 U.S.C. § 119(e) of U.S. Provisional Application Ser. No. 60/771,558, filed Feb. 8, 2006, the disclosure of which is incorporated herein by reference in its entirety.

### FIELD OF THE INVENTION

The field of this invention relates in general to compositions and methods useful for the study of protein interactions with other biomolecules, and more particularly to methods and compositions providing for the covalent capture on or immobilization to a support matrix of a protein covalently cross-linked to another biomolecule.

### BACKGROUND

The large scale and detailed study of proteins, particularly their functions and interactions, has been labeled “proteomics” and is widely viewed as a key to understanding the biochemistry of a cell. It has been determined through the Human Genome Project that humans may have between 20,000 and 30,000 protein coding genes, but that there may be between 100,000 and 400,000 proteins in the human proteome. The vast protein diversity generated from the limited number of protein coding genes is believed to be the result of alternative gene splicing and/or post-translational modifications. Because any organism will have different protein expression profiles in different cells, tissues, stages of its life cycle and/or under different environmental conditions, it may not be sufficient to merely understand the function of each of these proteins in isolation. To fully understand the biochemistry of the cell and the cellular processes necessary for the cell to perform its many functions requires an understanding of how expressed proteins interact with other proteins, nucleic acids or other biomolecules in the cell.

Various methods are employed to analyze interactions between a protein and another biomolecule. For example, transient protein:protein interactions may be detected and/or analyzed through use of various bait-prey models including the yeast two-hybrid system or by in vitro methods such as co-immunoprecipitation, cross-linking reagents, label transfer, protein arrays/protein chips, pull-down assays, nuclear magnetic resonance, mass spectroscopy and X-ray crystallography. Protein:nucleic acid interactions and complexes may be detected and analyzed by utilizing nucleic acid sequences labeled with an amine or biotin tag via a cross-linker that permits immobilization and subsequent detection.

Because many interactions between biomolecules are transient and occur for only a brief period of time sufficient to permit the biochemical function of the interaction, e.g. signaling or metabolic function, it is often difficult to capture the biomolecules during the interaction. The active complex is typically short-lived and often linked through non-covalent interactions, e.g., hydrogen, ionic, and/or other non-covalent forces, during the period of interaction. Through the use of cross-linking reagents, the active complexes themselves may be trapped in a covalently cross-linked complex sufficiently stable for isolation and characterization. Such a method does not, however, provide a means for an efficient capture of these complexes as available methods of capture are dependent upon non-covalent interactions with ligands on a solid support system, e.g., streptavidin/biotin systems. In such non-covalent capture systems, the trapped complex is often diluted or lost as a result of the need to perform extensive wash steps to remove non-specific interactions resulting from the cross-linking and the binding affinity of the support matrix itself. Thus, it would be an advance in the art to provide a capture system that does not interfere with the active complex while allowing for a covalent capture of the cross-linked complex to a support matrix to form a complete covalent bridge of attachment that can withstand rigorous purification such as repeated wash steps and/or further processing steps.

### SUMMARY OF THE INVENTION

Methods, compositions and kits to capture cross-linked protein complexes to a support matrix in a stable, covalent bridge of attachment are provided. It has been surprisingly discovered that a more effective, selective and robust capture of cross-linked protein:biomolecule complexes is achieved by utilizing a support matrix comprising a covalently attached ligand that in turn can covalently capture a cross-linked protein:biomolecule complex thereto.

The invention provides a method for capturing a target biomolecule that forms a complex in the presence of an interacting partner from a sample. The method includes providing a support matrix having at least one ligand covalently coupled thereto, the ligand capable of selective covalent attachment to a ligand-corresponding protein; forming a capture complex comprised of the target biomolecule, the interacting partner and the ligand-corresponding protein; treating the capture complex with a covalent cross-linking agent to form a covalently cross-linked capture complex; contacting the covalently cross-linked capture complex with the support matrix under conditions permitting the covalent attachment of the capture complex to the ligand. Alternatively, the capture complex may be combined with the support matrix and subsequently treated with a covalent cross-linking agent to form a covalently cross-linked capture complex attached to the support matrix; or the capture complex may be formed of the support matrix, the target biomolecule, the interacting partner and the ligand corresponding protein and then treated with the covalent cross-linking agent to form a covalently cross-linked capture complex.

The invention further provides a method for capturing and selectively releasing one member of a protein:protein interaction complex. The method includes providing a support matrix having at least one ligand covalently coupled thereto, the ligand capable of selective covalent attachment to a ligand-corresponding protein; forming a capture complex comprised of the protein:protein interaction complex and the ligand-corresponding protein; treating the capture complex with a reversible cross-linking agent to form a covalently cross-linked capture complex wherein the protein:protein interaction complex is covalently cross-linked in a manner covalently trapping the protein:protein interaction complex; contacting the covalently cross-linked capture complex with the support matrix under conditions permitting the covalent capture of the covalently cross-linked capture complex to the ligand through the ligand-corresponding protein; washing the support matrix having the captured capture complex to remove any unwanted biomolecules; and exposing the washed support matrix having the captured capture complex to conditions reversing the covalent cross-linking of the protein:protein interaction complex to allow the release of one member of the protein:protein interaction complex from the support matrix.

Also provided is a method for capturing and selectively releasing one member of a protein:nucleic acid interaction complex. The method includes providing a support matrix having at least one ligand covalently coupled thereto, the ligand capable of selective covalent attachment to a ligand-corresponding protein; forming a capture complex comprised of the protein:nucleic acid interaction complex and the ligand-corresponding protein; treating the capture complex with a reversible cross-linking agent to form a covalently cross-linked capture complex wherein the protein:nucleic acid interaction complex is covalently cross-linked in a manner covalently trapping the protein:nucleic acid interaction complex; contacting the covalently cross-linked capture complex with the support matrix under conditions permitting the covalent capture of the covalently cross-linked capture complex to the ligand through the ligand-corresponding protein; washing the support matrix having the captured capture complex to remove any unwanted biomolecules; and exposing the washed support matrix having the captured capture complex to conditions reversing the covalent cross-linking of the protein:nucleic acid interaction complex to allow the release of one member of the protein:nucleic acid interaction complex from the support matrix.

In one embodiment, the target biomolecule is nucleic acid and the interacting polypeptide is a fusion protein of a nucleic acid binding protein, such as a transcription factor, and a ligand-corresponding protein. In one embodiment, the fusion protein is expressed in mammalian cells. In one embodiment, the fusion, which includes a transcription factor, is expressed in mammalian cells and complexes are formed by the binding of the transcription factor to a transcription factor binding sequence in the genome of the mammalian cells. The complexes which are formed are cross-linked in vivo with, for instance, formaldehyde. The cells are lysed and sonicated to obtain small fragments of cross-linked chromatin. The cross-linked complexes are isolated on a support matrix, e.g., a resin such as a magnetic resin having a ligand for the ligand-corresponding protein. The resin is washed stringently to remove all non-specific complexes, including but not limited to DNA, protein, and protein:DNA complexes. The ligand-corresponding protein retains its activity after treatment with the cross-linking agent The crosslinks on the resin between the fusion and the nucleic acid are reversed, thereby releasing all nucleic acid fragments bound by the transcription factor in the fusion. The resulting fragments may be purified and concentrated for analysis. In one embodiment, a sample comprising mammalian cells expressing the fusion is placed in two receptacles. For the control sample, the ligand is added before isolation on a support matrix, thereby blocking the capture. The control sample indicates the amount of background nucleic acid isolated.

In one embodiment, to verify binding sites for a nucleic acid binding protein on the isolated fragments, nucleic acid amplification, such as PCR, quantitative PCR, real time PCR, such as Plexor™ based amplification, may be employed. In one embodiment, to identify sites for a nucleic acid binding protein, microarray/chip analysis (ChIP-on-chip) may be employed. To obtain sufficient nucleic acid for chip based analyses, ligation mediated-PCR (LM-PCR) followed by Cy3 and Cy5 labeling of control and experimental samples, respectively, may be employed.

In one embodiment, the invention provides a method for detecting the interaction of a polypeptide with a specific nucleic acid sequence. The method includes providing a support matrix having at least one ligand covalently coupled thereto, said ligand capable of selective covalent attachment to a ligand-corresponding protein; forming a complex comprised of the polypeptide and the ligand-corresponding protein; combining the complex with a nucleic acid sequence for a period of time and under conditions suitable for the polypeptide of the complex to bind to the nucleic acid sequence; treating the complex with a reversible cross-linking agent to form a covalently cross-linked complex wherein the polypeptide and the nucleic acid are covalently cross-linked; contacting the covalently cross-linked complex with the support matrix under conditions permitting the covalent capture of the covalently cross-linked capture complex to the ligand through the ligand-corresponding protein; washing the support matrix having the captured complex to remove any unwanted biomolecules; exposing the washed support matrix having the captured complex to conditions reversing the covalent cross-linking of the polypeptide-nucleic acid complex to allow the release of nucleic acid from the complex; and detection of resulting nucleic acid, preferably by nucleic acid amplification, more preferably by the polymerase chain reaction. Optionally, the solution containing the nucleic acid after release may be digested with a proteinase and/or the nucleic acid may be purified, for example, to concentrate the nucleic acid.

In further aspects, the general methods described above may be utilized in methods for capturing and selectively releasing one member of a protein:lipid interaction complex, a protein:carbohydrate interaction complex or a protein:small molecule complex, e.g. an organic molecule, from a biological sample in a manner as described.

In still further aspects, the invention provides compositions and kits for performing the methods described herein. In certain preferred embodiments, the cross-linking agent is a reversible cross-linking agent. In certain other preferred embodiments, the biological sample is a solution of biomolecules, a cell, a cell lysate or other biological fluid, e.g., blood, urine or tissue biopsy sample.

### BRIEF DESCRIPTION OF THE DRAWINGS

FIG. 1A provides an image of a gel subjected to electrophoresis, which compares the in vivo fluorescent labeling efficiency by the TMR ligand of HeLa cells transiently transfected with p65-HT.

FIG. 1B provides an image of a gel subjected to electrophoresis, which compares the in vivo fluorescent labeling efficiency by the TMR ligand of HeLa cells transiently transfected with CREB-HT.

FIG. 2 provides an image of a gel subjected to electrophoresis, which shows release of p65 and crosslinked p65 from HaloLink™ resin using Factor Xa.

FIG. 3A provides an image of a gel subjected to electrophoresis, which shows the amount of free TMR labeled p65-HT or CREB-HT after incubation for 2 hours with HaloLink™ resin.

FIG. 3B provides an image of a gel subjected to electrophoresis, which shows the amount of free TMR labeled p65-HT that have been stimulated by TNF-α after incubation for 2 hours with HaloLink™ resin.

FIG. 4 provides an image of an ethidium bromide stained agarose gel showing the PCR amplification of various human promoters from DNA fragments isolated after in vivo formaldehyde crosslinking.

FIG. 5A provides an image of an ethidium bromide stained agarose gel showing increased amplification of p65-specific promoters using in vivo protein:protein crosslinking followed by formaldehyde crosslinking.

FIG. 5B provides an image of an ethidium bromide stained agarose gel showing control PCR on samples of FIG. 5A using a promoter not known to interact with p65.

FIG. 6 provides an image of an ethidium bromide stained agarose gel showing increased amplification of p65-specific promoters after in vitro formaldehyde crosslinking.

FIG. 7 shows a schematic of the activation and attachment of Sepharose to a chloroalkane ligand.

### DETAILED DESCRIPTION OF THE INVENTION

It has been discovered that improved selectivity for the capture of cross-linked protein complexes obtained from in vivo or in vitro systems is achieved through the use of a composition and method that provides for a complete covalent bridge of attachment from a support matrix through the binding molecule and the molecule(s) being bound. By providing a capture mechanism of this nature, the trapped, cross-linked complex covalently bound to the support matrix may be subjected to more stringent wash procedures during the purification and isolation steps to remove undesired biomolecules allowing for a resulting complex that is more highly enriched for the complex containing the biomolecule of interest. For example, a mildly denaturing solution, such as 2M guanidinium thiocyanate with 1% CHAPS detergent, may be used to wash the matrix to remove non-specific background binding that would otherwise disrupt a non-covalent interaction by denaturing the proteins involved in the interaction. Other reagents such as guanidium chloride or urea may also be used and may be advantageous with a particular resin/support matrix. Because the present invention provides a covalently coupled bridge of attachment to the resin and has not reversed the crosslinks, such harsh conditions may be used. Moreover, samples containing small amounts of the target biomolecule may be utilized as less is needed to achieve detectable and analyzable results. Furthermore, through use of the method described herein, a significant savings in time for the researcher in capturing and isolating a desired biomolecule is achieved as compared to prior isolation and purification methods.

The present invention, in at least one embodiment, is directed to the isolation and/or identification of a biomolecule that is involved in a biochemical interaction with at least one other biomolecule. The biomolecule may be a protein, nucleic acid, lipid, carbohydrate, small molecule, or other chemical compound(s) of interest and combinations thereof. The biomolecule may be an individual chemical entity, e.g., a peptide, polypeptide, protein, lipid, carbohydrate, nucleic acid, small organic molecule or it may be a complex of such chemical entities such as a protein-protein complex, a protein-nucleic acid complex, a protein-lipid complex or a protein-small molecule complex. Thus, as used herein a biomolecule refers to at least one member of a biochemical interaction, e.g. involved in the function of a cell. Because protein interactions may have many functional objectives in a cellular environment, the biomolecule may be involved in such activities as altering the kinetic properties of an enzyme, allowing a substrate or cofactors to move between subunits, creating a new binding site, inactivating or destroying a protein, changing the specificity of a protein for its substrate, or serving a regulatory role in either an upstream or downstream activity.

In order to capture and subsequently analyze the biomolecule of interest (the target biomolecule) from a sample, its corresponding interacting partner is presented in a manner that permits the interaction to occur while also providing a mechanism that permits the resulting complex to be captured. As used herein, the interacting partner refers to the known entity to which the assay is looking for an interaction with the target biomolecule. The target biomolecule may be known to interact with the selected interacting partner and the assay determines if it is present in a particular sample or the target biomolecule may be unknown and the assay determines which if any biomolecule in a particular sample interacts with the interacting partner.

In accordance with the present invention, capture and analysis of a target biomolecule is achieved in one embodiment by forming or permitting the formation of a capture complex comprised of the target biomolecule, its interacting biomolecule and a ligand-corresponding protein. As used herein, a ligand corresponding protein refers to protein molecules that form a specific and selective covalent bond with a ligand or class of ligands upon their interaction. In a preferred embodiment, the ligand corresponding protein is a self-labeling protein tag that labels itself in the presence of its ligand, typically a low molecular weight compound, in a covalent manner. Preferred examples of self-labeling protein tags include mutant hydrolase compositions, such as the HaloTag® product of Promega Corporation Madison, Wis. USA as described in US20040002607, US2003000444094P, and US2003000474659P (the entirety of which are hereby incorporated herein by reference hereto), which utilizes a mutant dehalogenase protein that covalently couples a halo containing ligand thereto; the SNAP-tag® product of Covalys Biosciences, Switzerland, which is based on the human protein alkylguanine-DNA-alkyltransferase (AGT) protein which specifically transfers an alkyl residue from the O6 position of guanine to a reactive cysteine in the AGT molecule in a covalent manner as described in WO2002083937, PCT/EP03/10889 and PCT/EP03/10859, the entirety of each being hereby incorporated by reference hereto; and the cutinase/phosphonate covalent interaction as described by Hodneland, C., et. al, “Selective immobilization of proteins to self-assembled monolayers presenting active site-directed capture ligands” PNAS, vol. 99, no. 8, pp 5048-5052, Apr. 16, 2002, the entirety of which is hereby incorporated by reference hereto. In a preferred embodiment, the ligand corresponding protein is a modified dehalogenase enzyme from Rhodococcus rhodochrous that is commercially available from Promega Corporation as HaloTag®, which has specificity for a class of halo containing ligands, wherein the modification to the enzyme permits the formation of a covalent bond between the modified dehalogenase and the halo containing ligand when they are brought together in a reaction.

The ligand corresponding protein and the interacting partner are brought together such that it functions both to covalently couple to the ligand on the support matrix and to interact with the target biomolecule of interest, if present. The ligand corresponding protein and the interacting partner may be brought together in a covalent manner by any suitable means known in the art. In one embodiment, the ligand corresponding protein and the interacting partner are both proteins/polypeptides and are prepared as a fusion molecule by in vivo or in vitro expression, e.g., a fusion protein expressed from a recombinant DNA which encodes the ligand corresponding protein and at least one interacting protein of interest or a fusion protein formed by chemical synthesis. For example, the fusion protein may comprise a ligand corresponding protein, such as the modified dehalogenase described above and a channel protein, a receptor, a membrane protein, a cytosolic protein, a nuclear protein, a structural protein, a phosphoprotein, a kinase, a signaling protein, a metabolic protein, a mitochodrial protein, an immunomolecule, a receptor associated protein, an enzyme substrate, or other molecule. The protein of interest may be fused to the N-terminus or the C-terminus of the ligand corresponding protein. Optionally, the proteins in the fusion molecule may be separated by a connector sequence, e.g., preferably one having at least 2 amino acid residues, such as one having 13 to 17 amino acid residues, and the presence of a connector sequence does not substantially alter the function of either protein in the fusion relative to the function of each individual protein. Thus, the presence of a connector sequence does not substantially alter the stability of the bond formed between the ligand corresponding protein and the ligand thereof or the activity of the interacting protein. For any particular combination of proteins in a fusion, a wide variety of connector sequences may be employed. In one embodiment, the connector sequence is a sequence recognized by an enzyme, e.g., a cleavable sequence. For instance, the connector sequence may be one recognized by a caspase, e.g., DEVD, or is a photocleavable sequence.

In one embodiment, the fusion protein may comprise an interacting protein/polypeptide of interest at the N-terminus and, preferably, a different interacting protein/polypeptide of interest at the C-terminus of the ligand corresponding protein. The ligand corresponding protein and the interacting partner may be synthetically made by known chemical methods, and this is particularly suitable if the interacting partner is a nucleic acid, lipid or small molecule.

Thus, once the ligand-corresponding protein and the interacting partner molecule is formed it may be combined with the sample putatively containing the target biomolecule to for a capture complex. The resulting capture complex includes the target biomolecule, the ligand corresponding protein and the interacting partner, and is treated with a covalent cross-linking agent to form a covalently cross-linked capture complex. The treatment of the capture complex with the covalent cross-linking agent may be performed either prior to, simultaneous with or subsequent to contacting the capture complex with a support matrix that covalently binds the capture complex to the support matrix through its ligand. Thus, in one embodiment, the ligand corresponding protein and the interacting partner are expressed in a cell or introduced into a cell lysate for a period of time sufficient to permit the interacting partner to interact with the target biomolecule and to trap the molecules in the capture complex, and a suitable cross-linking agent is added.

The covalent cross-linking agent of the present invention is a composition that is capable of forming a covalent bond between any of the types of interactions between the interacting partner and the target biomolecule. For example, the cross-linking agent may form a covalent bond between an interacting pair comprised of a protein:DNA pair, a protein:protein pair, a protein:lipid pair, a protein:carbohydrate pair, a protein:small molecule pair or a DNA:small molecule pair. Any suitable cross-linking agent may be used including those that utilize as a basis of reactivity a chemical moiety including, but not limited to an amine, a sulfhydryl, a carbohydrate, a carboxyl, or a hydroxyl group. The cross-linking agent may be homobifunctional, having two identical reactive groups, and used in a one-step crosslinking reaction or may be heterobifunctional, having two or more different reactive groups, permitting sequential crosslinking reactions to improve specificity. The cross-linking agent may optionally be reversible or cleavable by any suitable cleaving mechanism, such as by addition of a thiol, a base, a periodate, or a hydroxylamine containing composition. As used herein, a cleavable or reversible cross-linking agent means such an agent that permits the cross-linking to be unformed or broken apart upon particular chemical treatment. The cross-linking agent may also optionally be iodinatable, membrane permeable, and/or water soluble. More preferably, suitable cross-linking agents include: formaldehyde (preferable for protein:DNA complexes, but also suitable to induce protein:protein crosslinks). Formaldehyde is available from many sources. Other cross-linking agents include bis(Sulfosuccinimidyl)suberate (BS3), a protein-protein non-reversible crosslinker (Pierce Biotechnology, Rockford Ill.); 3,3′-dithiobis(sulfosuccinimidylpropionate) (DTSSP), a protein-protein thiol-reversible crosslinker (Pierce Biotechnology, Rockford Ill.); and disuccinimidyl glutarate (DSG), a membrane permeable, non-cleavable, protein-protein crosslinker (Pierce Biotechnology, Rockford Ill.). In one embodiment, the cross-linking agent is reversible or cleavable upon treatment with a specific reversal/cleaving agent such that the target biomolecule may be removed from the capture complex and may be further isolated and/or purified for further analysis.

In some circumstances, certain amino acid(s) having desired functional groups are positioned at useful locations in the peptide to permit the desired cross-linking to occur. Alternatively, such amino acid(s) may be introduced at specified positions within the peptide. In addition, it is preferable that such functional groups be present only once per nucleic acid-protein fusion molecule, or be positioned in a way such that a higher relative reactivity in cross-link formation, compared to potential competitors, is established. This could be achieved, for example, by employing nucleic acid hybridization methods to position the desired reactive groups such that a specific cross-linking reaction is promoted.

The selection of a suitable cross-linking agent is known to one skilled in the art and is typically selected on the basis of their chemical reactivities (i.e., specificity for particular functional groups) and compatibility of the reaction with the desired application. The preferred cross-linking agent to use for a specific application may be determined empirically and may be chosen based on chemical specificity, spacer arm length, reagent water-solubility and cell membrane permeability, whether the same (homobifunctional) or different (heterobifunctional) reactive groups are preferred, the need for thermoreactive or photoreactive groups, whether the reagent cross-links are cleavable or not, or whether the reagent contains moieties that can be radiolabeled or tagged with another label.

Cross-linkers contain at least two reactive groups. Functional groups that can be targeted for cross-linking include primary amines, sulfhydryls, carbonyls, carbohydrates and carboxylic acids. Coupling also can be nonselective using a photoreactive phenyl azide cross-linker. Often different spacer arm lengths are required because steric effects dictate the distance between potential reaction sites for cross-linking. For protein:protein interaction studies, a cross-linker with a short spacer arm (4-8 Å) may be used and the degree of cross-linking determined. A cross-linker with a longer spacer arm may be used to optimize cross-linking efficiency. Short spacer arms are often used in intramolecular cross-linking studies, and intermolecular cross-linking is favored with a cross-linker containing a long spacer arm.

In some applications, to preserve the native structure of the protein complex, cross-linking may be performed using mild pH and buffer conditions, e.g., physiological pH and buffer conditions. Optimal cross-linker-to-protein molar ratios for reactions may be determined. If there are functional groups, a lower cross-linker-to-protein ratio may be used. For a limited number of potential targets, a higher cross-linker- to-protein ratio may be employed.

The present invention utilizes a support matrix onto which the ligand that covalently couples to the ligand-corresponding protein is itself covalently coupled. The support matrix may be any substrate suitable for use in connection with the isolation, capturing and/or purification of biomolecules and includes the use of inorganic crystals, inorganic glasses, inorganic oxides, metals and/or polymers including but not limited to a resin such as a magnetic resin, a hydrogel and/or polymer hydrogel array. Preferably, the support matrix is formed of a polymeric material. Suitable polymers can be any polymer or mixture of polymers, including but not limited to, hydrophilic polymers. More particularly, the support matrix may include one or more of the following polymers, polyamide, polyacrylamide, polyester, polycarbonate, hydroxypropylmethylcellulose, polyvinylchloride, polymethacrylate, polystyrene and copolymers of polystyrene, polyvinyl alcohol, polyacrylic acid, polyethylene oxide and combinations thereof. The support matrix can also be formed of one or more of the following substances, collagen, dextran, cellulose, cellulosics, calcium alginate, latex, polysulfone, agarose, including but not limited to cross-linked agarose products, such as Sepharose® (GE Healthcare) and its various embodiments, and glass. In one embodiment, the support matrix includes paramagnetic agarose particle(s).

The support matrix may take any suitable shape or arrangement so long as the ligand remains exposed or available for reaction with a ligand-corresponding protein. In one embodiment, the support matrix is a highly cross-linked agarose matrix such as Sepharose 4B® (GE Healthcare) onto which the ligand has been covalently attached. The support matrix can be provided as a separate entity or as an integral part of an apparatus, e.g. a bead, cuvette, plate, vessel, column or the like.

The ligand is covalently bound to the support matrix in a manner exposing its reactive moiety for the ligand corresponding protein. As is understood, the choice of ligand corresponds to the choice of ligand-corresponding protein utilized in the capture complex. In one embodiment the ligand and the ligand-corresponding protein present chemical moieties to covalently attach through the formation of an ester bond or a thioether bond. For example, if a Halotag® ligand-corresponding protein is utilized the ligand may be an alkylhalide, e.g., a chloroalkane presenting the chloro-group available for interaction/covalent coupling and the chloroalkane ligand covalently couples to the mutant dehalogenase via an ester bond. If the ligand corresponding protein is a SnapTag molecule, the ligand may be a para-substituted benzyl guanine and these molecules covalently couple through a thioether bond. If the ligand corresponding protein is cutinase, the corresponding ligand may be a phosphonate that mimics the tetrahedral transition state of an ester hydrolysis. The selected ligand is coupled to the support matrix by known chemical methods. In an alternate embodiment, a plurality of different ligands may be introduced on the support matrix to permit different combinations of ligand corresponding proteins to be used to capture different target molecules in a single assay.

The following examples are intended as illustrations of what may be practiced by using the present kits, methods and compositions. They are not intended to be limiting, and any person skilled in the art would appreciate the equivalents embodied therein.

### EXAMPLES

For demonstration of the concepts of the invention, the following experimental materials and methods were used with modifications in particular examples noted. In all cases, the term “bait” will be used for the protein which is covalently captured to the solid support system, while the term “prey” will be used for the biomolecule that is cross-linked to the bait. The solid support system used in these experiments is HaloLink™ resin (Promega), which contains a chloroalkane ligand attached to Sepharose particles. The chloroalkane ligand covalently binds to HaloTag® (HT), a mutant dehalogenase protein. Therefore, specific examples of Bait proteins used for these experiments include C-terminal HT fusions of the following: Jun-HT, Fkbp-HT, Frb-HT, p65-HT, and CREB-HT, and HT alone as a control. Proteins used as Prey include: GST-cFos, GST-Frb, and GST-Fkbp, and purified mammalian HeLa genomic DNA.

All proteins used in these studies were cloned into mammalian and in vitro expression Flexi-vectors (Promega). Flexi-vectors, pFC8A and pFN2A encode C-terminal HT and N-terminal GST fusion proteins, respectively. Proteins used for in vitro experiments were expressed in Rabbit Reticulocyte Lysate Transcription/Translation-coupled cell free expression systems (Promega). For detection and visualization purposes, in vitro expressed Prey proteins were fluorescently labeled using Lys-tRNA with FluoroTect Green (Promega), and Prey DNA was amplified using Polymerase Chain Reaction (PCR) and visualized with ethidium bromide. Detection of Bait and/or Prey however is not limited to these methods and can be achieved by a variety of methods known to those skilled in the art.

HeLa cells used for in vivo crosslinking experiments were cultured in DMEM supplemented with 10% Fetal Bovine Serum (Gibco). Cells transfected with p65-HT were stimulated with the addition of recombinant purified TNF-α (Sigma #T0157) and analysed by Western blots using p65 antibody (BD Biosciences #610868). DyLight fluorescent protein markers (Pierce) and Benchtop 100 bp DNA ladder markers (Promega) were used for protein and DNA electrophoresis gels.

Protein-protein crosslinkers used for in vitro and in vivo crosslinking include DTTSP and DGS, respectively (Pierce). DTTSP was prepared at a stock concentration of 10 mM in 1×PBS, and DGS at 1 mM in DMSO.

PCR primers used to amplify corresponding human HeLa promoter sequences include: IL-8 (position −121 relative to the initiator AUG start codon) 5′-GGGCCATCAGTTGCAAATC-3′ (SEQ ID NO:1) and (+61) 5′-TTCCTTCCGGTGGTTTCTTC-3′ (SEQ ID NO:2), ICAM (−339) 5′ GGTTGGCAGTATTTA-3′ (SEQ ID NO:3) and (−174) 5′-GCCTCGCTGGCCGCT-3′ (SEQ ID NO:4), IKβα 5′-GACGACCCCAATTCAAATCG-3′ (SEQ ID NO:5) and 5′-TCAGGCTCGGGGAATTTCC-3′ (SEQ ID NO:6), GAPDH 5′-TACTAGCGGTTTTACGGGCG-3′ (SEQ ID NO:7) and 5′-TCGAACAGGAGGAGCAGAGAGCGA-3′ (SEQ ID NO:8), CNAP 5′-ATGGTTGCCACTGGGGATCT-3′ (SEQ ID NO:9) and 5′-TGCCAAAGCCTAGGGGAAGA-3′ (SEQ ID NO:10), and hCGα (−213) 5′-GTCGTCACCATCACCTGAAAA-3′ (SEQ ID NO:11) and (−34) 5′-CAGAGTGTTTCCACCTGCAT-3′ (SEQ ID NO:12). GoTaq green master mix (Promega) was used for all PCR experiments.

In vitro Protein:Protein Crosslinking After in vitro protein expression, approximately 50-100 ng of both Bait and Prey are mixed by rotation at 22° C. for one hour. For enhanced crosslinking of proteins expressed in in vitro lysates, an equal volume of 2× Phosphate Buffered Saline (PBS) may be added to the Bait-Prey mixture. Add crosslinker to a final concentration of 1-5 μM, as recommended by the manufacturer for the particular concentration of proteins, and incubate for 30 minutes at 22° C. The crosslinking reaction is quenched by the addition of Tris pH 7.5 to a final concentration of 20 mM incubate for 15 minutes at 22° C.

Covalent capture of in vitro crosslinked protein:protein complexes on solid support system. Binding to HaloLink™ resin is performed as published in Promega HaloLink™ Technical Manual TM250. Any deviations from this protocol are described below. For 100-200 ng of cross-linked complex, aliquot 5011 of a 25% ethanol slurry of HaloLink™ resin. Centrifuge for 1 minute at 800×g and remove the ethanol. Wash resin 3×400 μl with 1×TBS+0.05% IGEPAL using repeated centrifugation at 800×g. Remove final wash solution and add protein-protein crosslinked material to resin. Incubate at 22° C. for one hour. Wash resin 5×1 mL with 1×TBS+0.05% IGEPAL as described above. Remove final wash. At this point, the crosslinked protein-protein complex has been covalently captured and purified on the resin and can be resuspended in the desired buffer. In samples treated with the crosslinker DTTSP, resupend resin in 1×SDS Loading buffer containing 5 mM β-Me and boil at 95° C. for 5 minutes to reverse the protein-protein crosslinks. Analyse samples using SDS gel electrophoresis.

Covalent Capture of Bait Protein followed by crosslinking to Prey. Prepare 50 μl of HaloLink™ resin slurry and remove ethanol as described above. Wash resin 3×400 μl with 1× Phosphate Buffered Saline (PBS)+0.05% IGEPAL. Remove final wash solution and incubate resin with approximately 50-100 ng of Bait protein for 30-60 minutes at 22° C. Add 50-100 ng of prey, optionally along with an equal volume of 2×PBS, to the resin-Bait mixture and incubate at 22° C. for 30-60 minutes. Add cross-linker to a final concentration of 1-5 μM and incubate for 30 minutes at 22° C. Quench reaction as described above. Wash resin 5×1 mL with 1×TBS+0.05% IGEPAL and if using a thiol-reversible crosslinker identify prey as previously stated.

In vitro Protein:DNA Crosslinking Capture Bait protein on HaloLink™ resin as described above. Wash resin 3×1 mL with 20 mM HEPES pH 7.0, 150 mM NaCl, 5 mM MgCl₂, 0.05% IGEPAL. Add purified, sonicated genomic DNA (Prey) at 2× molar concentration. Incubate for 15 minutes at 22° C. Add formaldehyde to a final concentration of 0.75% and mix for 30 minutes at 22° C. Wash resin 3×1 mL with 1×TBS+0.05% IGEPAL, followed by 3×1 mL washes using 2 M GuSCN pH 7.5+1% CHAPS. Wash resin 1×1 ml with 20 mM Tris pH 6.8, 300 mM NaCl, and 10 mM EDTA and resuspend in 100 μl of this same buffer. Incubate at 65° C. for 4-5 hours to reverse crosslinks. Purify DNA using a Wizard SV Gel and PCR Clean-up Kit (Promega) and identify DNA fragments using standard PCR.

In vivo Protein:DNA Crosslinking Transfect 1×10⁶ mammalian cells at 80-90% confluency with 1 μg of Bait protein and Lipofectamine 2000 (Invitrogen). At 16-24 hours post-transfection, add formaldehyde to a final concentration of 1% directly to media. Incubate for 10 minutes at 22° C. Stop crosslinking by the addition of glycine to a final concentration of 125 mM. Incubate for 5 minutes at 22° C. Wash cells twice with ice-cold 1×PBS. In one example, Bait proteins were fused to HT, and could therefore be detected by staining with a membrane-permeable, covalent, fluorescent ligand termed TMR (Promega). For TMR staining, incubate cells with 5 μM of TMR diluted directly into supplemented media for 10 minutes at 37° C. TMR staining was performed either before or after formaldehyde treatment. Wash cells twice with 1×PBS to remove excess TMR ligand. For samples analysed by SDS gel electrophoresis or other techniques including Western blotting, lyse cells by addition of 1×SDS loading buffer.

Covalent capture of in vivo crosslinked protein:DNA complexes, reversal of crosslinks, and identification of DNA fragments. Transfect cells, crosslink with 1% formaldehyde, and stop crosslinks as described above. Wash cells twice with ice-cold 1×PBS. Scrape cells from dish in ice-cold 1×PBS plus protease inhibitors and centrifuge at 800×g for 5 minutes. Resuspend pelleted cells in Lysis buffer (1% Triton X-100, 0.1% NaDOC, 150 mM NaCl, 5 mM EDTA, 20 mM Tris pH 8.0, protease inhibitors) and incubate on ice for 15 minutes. To ensure lysis is complete, cells can be dounced or pipetted through a 27 Gauge needle tip. Sonciate chromatin on ice to an average length of 200-1000 bp using a Misonix 3000 at a setting of 1.5 or 2, with a program of four 10 second pulses, followed by 10 seconds of rest. Pellet cell debris and clear lysates by centrifugation at 10,000×g for 10 minutes. At this juncture, lysates containing crosslinked complexes can be covalently attached to any type of solid support which the crosslinked protein complexes of interest might bind. In one example, Bait proteins were fused to HT, therefore crosslinked complexes were captured on HaloLink™ resin, prepared as described above. For 1×10⁶ cells, 75 μl of 25% HaloLink™ resin were used. Incubate lysates with HaloLink™ resin at 22° C. for 2-3 hours with mixing. Follow the same wash steps, crosslink reversal, and DNA purification protocol as used for in vitro protein:DNA crosslinking. Identify DNA fragments using standard PCR.

Covalent Capture of in vivo crosslinked protein:protein:DNA complexes. Transfect cells as described above. At 24 hours post-transfection, wash cells with 1×PBS+1 mM MgCl₂. Add the protein:protein crosslinker directly to cells at a final concentration of 2 mM in 1×PBS+1 mM MgCl₂. Incubate for 45 minutes at 22° C. Wash cells 3 times with 1×PBS. Add 1% formaldehyde in 1×PBS+1 mM MgCl₂ to cells and incubate for 15 minutes at 22° C. Stop crosslinking, wash cells, and isolate Bait-HT crosslinked samples following the protocol as described for in vivo protein:DNA crosslinking. Wash resin, reverse crosslinks, purify DNA, and perform PCR as described for in vitro protein:DNA cross linking.

### Example 1

### Covalent Capture of Crosslinked Bait:Prey Complexes

Fkbp and the Frb domain of Frap have been shown to form a complex in the presence of a small molecule, rapamycin¹. The minimal domains of Fkbp and Frb needed for the interaction¹ were expressed in vitro as HT and GST fusion proteins in cell-free lysates as described. The Prey protein, GST-Frb, was fluorescently labeled using Fluorotect for detection and mixed with the Bait, Fkbp-HT, as described. The Bait:Prey mixture was then combined with HaloLink™ resin (as described above), which covalently binds HT, in the presence or absence of rapamycin (2 μM) and/or the thiol reversible protein:protein crosslinker, 5 mM DTTSP. Table 1 below represents the gel electrophoresis results showing the release of amounts of fluorescently labeled GST-Frb from the HaloLink™ resin under the test conditions. The lane labeled SM (Starting Material) represents the fluorescently labeled GST-Frb alone. GST-Frb was incubated with HT alone (Table 1, Lane 1), resin alone (Table 1, Lane 2) or with Fkbp-HT (Table 1, Lanes 3-8). Rapamycin is required for the Bait:Prey interaction as shown in Lanes 4, 7, 8 as Prey is not detected in the absence of rapamycin (Lanes 3, 5, 6). Control experiments using either HT alone (Lane 1) or resin only with Prey showed no background binding of Prey, indicating that the interaction between Fkbp:Frb is specific (Table 1, Lanes 1, 2). Irrespective of the crosslinker, Bait:Prey complex formation is observed only in samples containing rapamycin (Table 1, Lanes 5-8), and an increased amount of Prey is released from the resin after reversal of the crosslink as described above (Table 1, Lane 8). This indicates that Bait:Prey crosslinked complexes were covalently captured on HaloLink™ and Prey was successfully released after reversal of the crosslink.

TABLE 1

| | | | | | | | | | |
|---|---|---|---|---|---|---|---|---|---|
| Rapamycin | | + | + | − | + | − | − | + | + |
| DTTSP | | − | − | − | − | + | + | + | + |
| Lane | SM | 1 | 2 | 3 | 4 | 5 | 6 | 7 | 8 |
| GST-Frb | ✓ | − | − | − | ✓ | − | − | ✓ | ✓ |

### Example 2

### Comparison of Crosslinking Bait:Prey Complexes Before or after Covalent Capture

The interaction between the transcription factors c-Jun and c-Fos is a well-characterized and is a high affinity interaction². Similar to Example 1, binding experiments were performed using HaloLink™ Resin with c-Jun-HT as Bait and fluorescently labeled GST-cFos (Table 2a, SM) as Prey. Table 2A shows the gel electrophoresis results of the release of fluorescently labeled Prey from: HaloLink™ resin under the conditions described. Bait and Prey were treated in the following conditions: a. crosslinked with 5 mM DTTSP, then captured on HaloLink™, b. captured on HaloLink™, then crosslinked with 5 mM DTTSP, c. or captured on HaloLink™ with no crosslinker present (Table 2A, Lanes 1-3 respectively). The capture and release of prey occurs without the presence of crosslinker (Table 2A, Lane 3), while the detection of prey in the presence of DTTSP from the samples in Lanes 1 and 2 is observed only when crosslinking is reversed (Table 2A, Lanes 4 and 5). This indicates that DTTSP is a very efficient crosslinker for this Bait:Prey complex and also shows that protein complexes can be either crosslinked then covalently captured, or covalently captured and crosslinked with similar results. The same experiments were performed using HT alone as Bait with GST-cFos as Prey and revealed no non-specific crosslinking of Prey to the Resin or HT either in pre of post-treatment with the crosslinker (Table 2B, Lanes 1-5).

TABLE 2A

| | | | | | | |
|---|---|---|---|---|---|---|
| DTTSP | | + | + | − | + | + |
| Lane | SM | 1 | 2 | 3 | 4 | 5 |
| GST-cFos | ✓ | − | − | ✓ | ✓ | ✓ |

TABLE 2B

| | | | | | | |
|---|---|---|---|---|---|---|
| DTTSP | | + | + | − | + | + |
| Lane | SM | 1 | 2 | 3 | 4 | 5 |
| GST-cFos | ✓ | − | − | − | − | − |

### Example 3

### Detection of In Vivo Crosslinked Protein:DNA Complexes

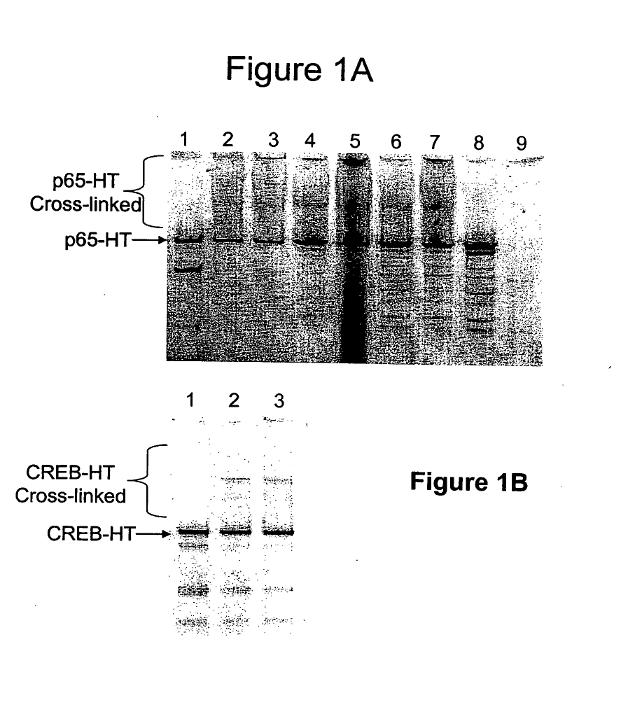

In order to determine whether or not in vivo crosslinked protein:DNA complexes would be able to covalently bind a ligand, i.e. the fluorescent TMR chloroalkane ligand (Promega Corp, Madison, Wis., Product #G8251), experiments were performed using two nuclear transcription factor proteins, p65 and CREB, expressed as HT fusion proteins. These proteins have been shown to bind to DNA at specific promoter regions and activate transcription in conjunction with a variety of other transcriptional proteins³. Both HT fusion proteins were transiently transfected into HeLa cells and treated with various concentrations of formaldehyde to induce protein:DNA crosslinks⁴ and subsequently lysed, either before or after labeling with the TMR ligand. The gel electrophoresis results for these experiments for p65-HT and CREB-HT are shown in FIGS. 1A and 1B, respectively. In FIG. 1A, lane 1 represents no formaldehyde added, lanes 2 and 4 represent 0.5% formaldehyde added, and lanes 3 and 5 represent the addition of 1% formaldehyde. The cells in lanes 2 and 3 were lysed before TMR labeling and the cells in lanes 4 and 5 were lysed after TMR labeling. Lanes 6 and 7 in FIG. 1A represent transfected cells that were TMR labeled followed by treatment with either 0.5% formaldehyde (lane 6) or 1% formaldehyde (lane 7). Lane 8 of FIG. 1A represents the results of reversing the crosslinking from samples of lane 7. Lane 9 of FIG. 1A is a control of untransfected HeLa cells labeled with TMR. FIG. 1B, lane 1 represents cells treated with no formaldehyde, lane 2 is 1% formaldehyde and lysed after TMR labeling. Lane 3 of FIG. 1B represents cells TMR labeled followed by a 1% formaldehyde treatment. The presence of crosslinked p65-HT and CREB-HT is indicated by a set of slower migrating upper bands during gel electrophoresis (FIG. 1A, Lanes 2-7, FIG. 1B, Lanes 2-3), as expected if they are crosslinked to various lengths of chromatin DNA⁴. The results also show that p65-HT and CREB-HT covalently bind the TMR ligand before (FIG. 1A, Lanes 6-7 and FIG. 1B, Lane 3) and after (FIG. 1A, Lanes 2-5a, and FIG. 1B, Lane 2) formaldehyde treatment in vivo. The crosslinks were reversed as described in the Methods, resulting in the loss of slower migrating bands (FIG. 1A, Lane 8). The control experiment using untransfected cells showed low background binding of the TMR ligand (FIG. 1A, Lane 9).

### Example 4

### Covalent Capture of In Vivo Crosslinked Protein:DNA Complexes on a Solid Support

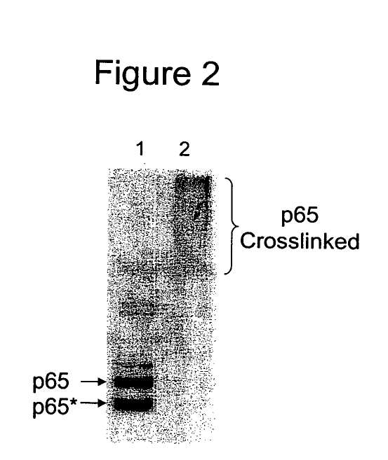

The p65-HT fusion protein contains a Factor Xa proteolytic cleavage site between p65 and HT, which upon binding to HaloLink™ resin, allows for release of p65 from the HaloLink resin after Factor Xa treatment. HeLa cells transfected with p65-HT were either treated without formaldehyde or with 1% formaldehyde, lysed, incubated with HaloLink™, and subjected to Factor Xa cleavage. The resulting supernatants containing p65 or p65 crosslinked species were analysed by Western blotting, using a primary antibody against p65 and a secondary HRP-conjugated antibody for detection. The results are shown in FIG. 2. Lane 1 of FIG. 2 represents non-crosslinked cells (no formaldehyde) and lane 2 represents crosslinked cells (1% formaldehyde). In cells not crosslinked with formaldehyde, p65 is released and shows some slight sensitivity in degradation after Factor Xa treatment (FIG. 2, Lane 1). However, in formaldehyde treated cells, the migration of p65 is significantly shifted upward (FIG. 2, Lane 2), indicative of the formation of higher molecular crosslinked species. The inability of the crosslinked complexes to migrate as a single band is consistent with the understanding that p65 is crosslinked to chromatin DNA of many different lengths. The smaller, proteolytic fragment produced by Factor Xa treatment is depicted as p65* in FIG. 2.

### Example 5

### Optimization of Covalent Capture of Crosslinked Protein:DNA Complexes

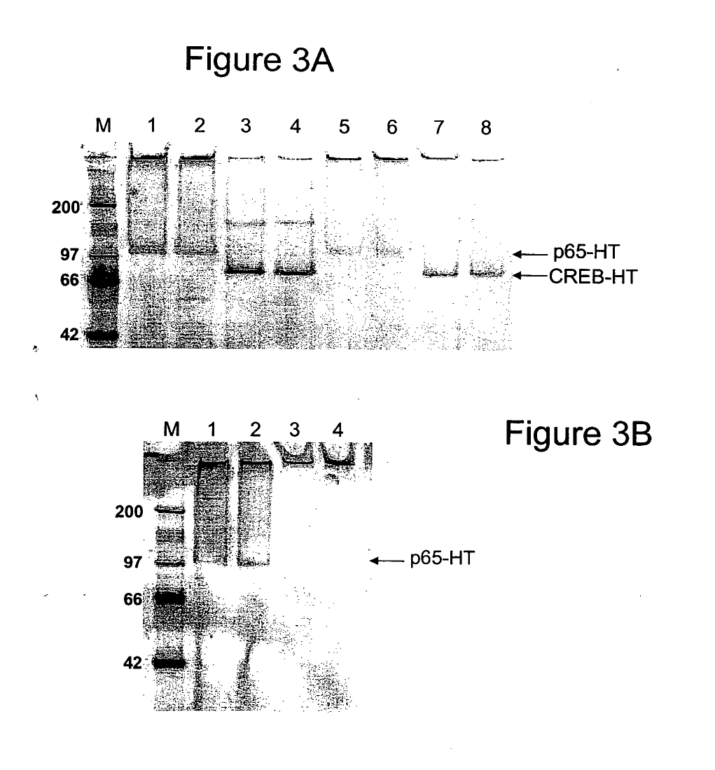

In order to optimize the efficiency for lysing formaldehyde treated cells, multiple lysis conditions were tested with the goal being to identify optimal lysis conditions, maintain chromatin solubility, and retain binding capacity to resin. FIGS. 3A and 3B are gel electrophoresis results showing the amount of free TMR labeled p65-HT and/or CREB-HT after incubation with HaloLink™ resin for 2 hours. In FIG. 3A, HeLa cells were transfected with p65-HT (lanes 1, 2, 5-6) or CREB-HT (lanes 3-4, 7-8), crosslinked with 1% formaldehyde, and lysed with either buffers containing 1% Triton X-100+0.1% NaDOC (lanes 1, 3, 5, 7) or 1% Triton X-100+0.1% Tomah (lanes 2, 4, 6, 8). Prior to HaloLink™ binding, aliquots of p65-HT (lanes 1-2) and CREB-HT (lanes 3-4) lysates were labeled with TMR. Lysates were incubated with HaloLink™ resin for 2 hours and aliquots of the supernatant (the unbound fraction) from both p65-HT (lanes 5-6) and CREB-HT (lanes 7-8) were labeled with TMR. In FIG. 3B, HeLa cells were transfected with p65-HT, stimulated by TNF-α (lanes 1-4) and also a protein:protein crosslinker DGS (lanes 2, 4). Aliquots of starting lysates (lanes 1, 2) and post HaloLink™ supernatants (lanes 3-4) were labeled with TMR as described above. All TMR labeled proteins were detected on the Typhoon Imager and intensity of bands were quantitated using ImageQuant. Molecular weights (kDa) of fluorescently labeled protein markers (M) are shown.

Detergent conditions consisting of 1% Triton+0.1% NaDOC or 1% Triton X-100+0.1% Tomah were found to meet the desired criteria when using HT fusion proteins (FIG. 3). The percentage of p65-HT and CREB-HT bound to HaloLink™ after 2 hours in 1% Triton X-100+0.1% NaDOC was 74% and 71% (FIG. 3A, Lanes 2, 4, 6, 8), while in 1% Triton X-100+0.1% Tomah was 65% and 66% respectively (FIG. 3A, Lanes 1, 3, 5, 7). An even higher percentage, 95% and 96%, of p65-HT was found to bind to HaloLink™ resin in 1% Triton X-100+0.1% NaDOC after stimulation with TNF-α (FIG. 3B, Lanes 1, 3) or in combination with the protein:protein crosslinker DSG (FIG. 3B, Lanes 2, 4). This indicates in vivo crosslinked complexes can be covalently attached to solid supports, and can be optimized for each system by routine experimentation.

### Example 6

### Identification of DNA Covalently Captured from In Vivo Crosslinked Protein:DNA Complexes

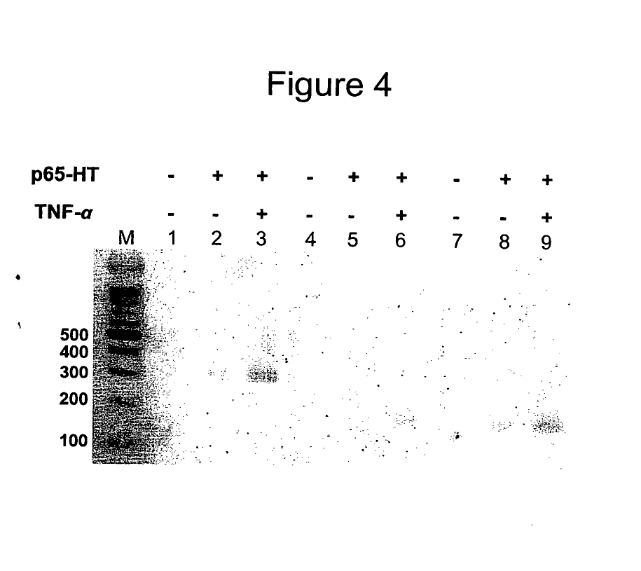

Promoter sequences for p65 have been identified and have been shown to be bound by endogenous p65 after in vivo formaldehyde crosslinking^{5,6}. Promoter binding by p65 increases after TNF-α stimulation, which promotes the translocation of p65 from the cytoplasm to the nucleus^{5,6}. Using this model, untransfected and p65-HT transfected cells were treated with or without TNF-α, crosslinked with formaldehyde, and bound to HaloLink™. The resin was stringently washed to remove all non-specific protein and DNA binding. Formaldehyde crosslinks were reversed, releasing genomic DNA fragments crosslinked to p65-HT. After purification of the DNA fragments, PCR was performed to determine if p65 specific promoters were amplified. FIG. 4 is an ethidium bromide stained 2% agarose gel showing the PCR amplification of various human promoters from DNA fragments isolated after in vivo formaldehyde crosslinking. As indicated in FIG. 4, HeLa cells were untransfected, or transfected with p65-HT, stimulated or not by TNF-α, crosslinked with 1% formaldehyde, lysed and sonicated to shear chromatin. Cleared lysates containing cross-linked protein:DNA complexes were incubated with HaloLink™, stringently washed with 2M Guanidinium thiocyanate with 1% CHAPS detergent (a mildly denaturing solution), and formaldehyde crosslinks were reversed. Released DNA fragments were purified and PCR amplified using primers for p65 specific promoter regions from the following gene targets: IKβα (300 bp) (lanes 1-3), IL-8 (182 bp) (lanes 4-6), and ICAM (165 bp) (lanes 7-9). DNA marker sizes are shown in the lane marked M.

FIG. 4 shows that three p65-specific promoter regions, IKβα, IL-8, and ICAM, were each amplified in cells transfected with p65-HT (FIG. 4, Lanes 2, 5, 8) compared to untransfected cells (FIG. 4, Lanes 1, 4, 7). Stimulation by TNF-α showed increased amplification of these promoter regions (FIG. 4, Lanes 3, 6, 9) compared to untransfected, and p65-HT with no TNF-α stimulation. IKβα, IL-8, and ICAM show a 22, 1.2, 4.5 fold amplification of the respective promoters compared to untransfected cells. Comparison of transfected cells versus transfected cells stimulated with TNF-α result in a 3.5, 3.2, 3 fold increase of activation. These increase of amplification are similar to previously reported values isolating endogenous p65 crosslinked to chromatin with or without TNF-α stimulation in Chromatin Immunoprecipitation (ChIP) studies^{5,6}.

### Example 7

### Identification of DNA Covalently Captured from In Vivo Crosslinked Protein:DNA and Protein:Protein:DNA Complexes

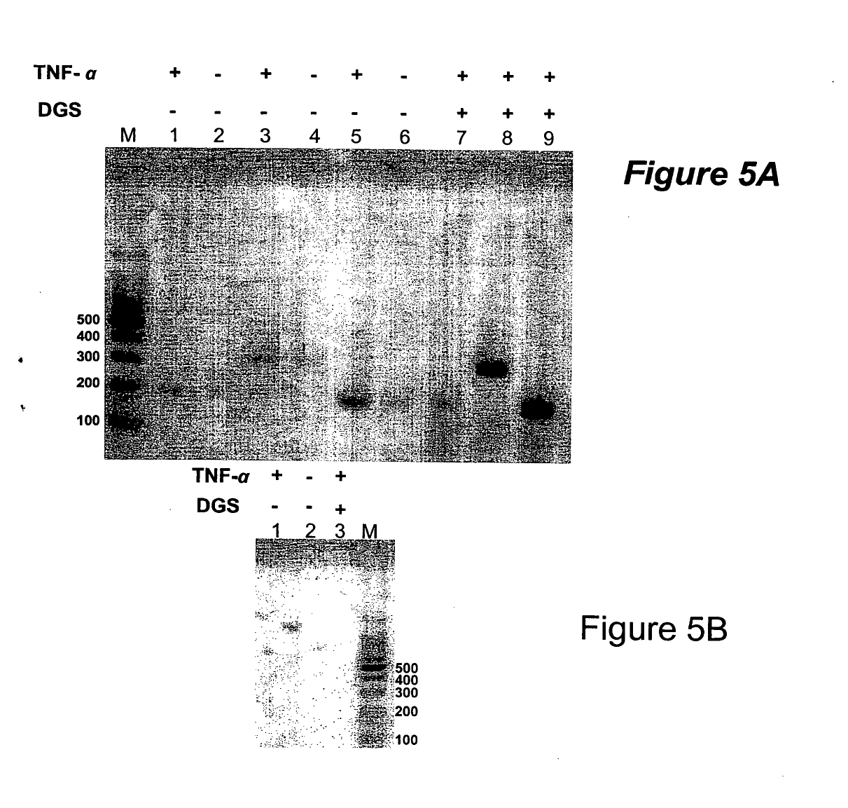

It has been shown that p65 interacts with several other transcription factors while binding to DNA⁴. The use of protein:protein crosslinkers in vivo has resulted in trapping of multi-protein complexes⁷. In attempts to trap transcription complexes containing p65 bound DNA, cells were treated with a protein:protein crosslinker prior to formaldehyde treatment⁶. Using the same protocol as outlined in Example 6, DNA fragments were isolated after treatment without crosslinkers, formaldehyde alone, or the combination of protein:protein plus formaldehyde, were PCR amplified and the results are shown in FIG. 5. Images of ethidium bromide stained 2% agarose gels showing increased amplification of p65 specific promoers using in vivo protein:protein crosslinking followed by formaldehyde crosslinking are shown in FIGS. 5A and 5B. As indicated in FIG. 5A, HeLa cells were transfected with p65-HT and stimulated with or without TNF-α. Lanes 7-9 were treated with 5 mM DGS crosslinker prior to protein:DNA formaldehyde crosslinking. All other samples were crosslinked with formaldehyde only, and DNA used for PCR was isolated as described in Example 6. The p65 specific promoter regions amplified via PCR were IL-8 (182 bp) (lanes 1, 2, 7), IKβα (300 bp) (lanes 2, 3, 8) and ICAM (165 bp) (lanes 5, 6, 9). In FIG. 5B, control PCR was performed on te same samples using a promoter not shown to interact with p65, CNAP (172 bp), lanes 1-3. DNA molecular weight markers are shown in the lane labeled M.

The results show that amplification of two p65-specific promoters, IKβα and ICAM, are both increased by 3 fold upon treatment with both protein:protein and protein:DNA crosslinkers, and TNF-α (FIG. 5A, Lanes 3-6 compared to Lanes 8, 9). IL-8 amplification remains the same (FIG. 5A Lane 1 compared to Lane 7). As a control, a promoter region that p65 does not bind, CNAP, was not amplified in any of the conditions, indicating, that crosslinking of p65-HT is specific (FIG. 5B).

### Example 8

### Covalent Capture of Bait Protein Followed by In Vitro Crosslinking to DNA

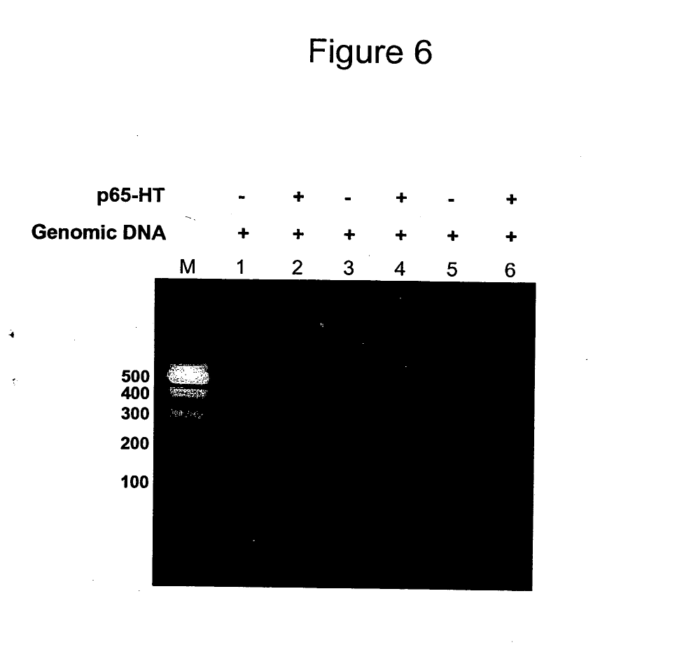

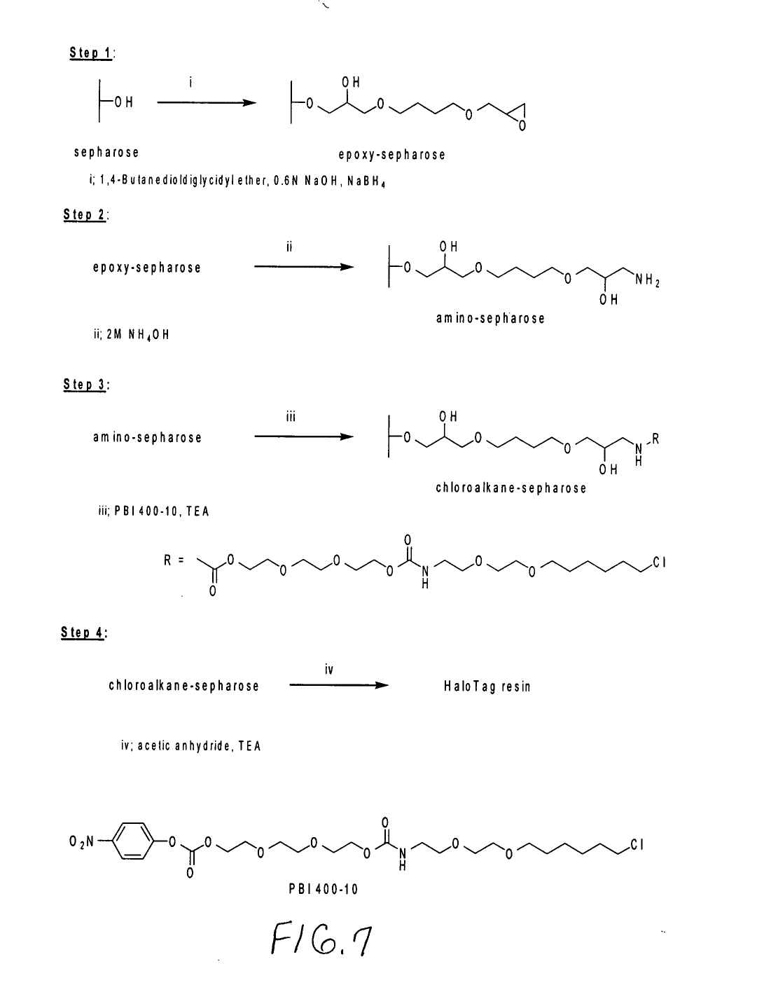

As previously described, human p65 target promoter sequences have been identified^{5,6}. To determine if protein:DNA crosslinking could occur in vitro, p65-HT was bound to HaloLink™ resin (a Sepharose based resin, see FIG. 7), washed, and crosslinked with formaldehyde to sheared, purified genomic HeLa DNA. Following similar protocols for crosslink reversal and DNA purification as described in Example 6, p65 specific promoter regions were PCR amplified from isolated DNA fragments. As a control, genomic DNA was crosslinked to resin alone, purified, and subjected to the same PCR conditions. The results are shown in FIG. 6 which is an ethidium bromide stained 2% agarose gel showing increased amplification of p65-specific promoters after in vitro formaldehyde crosslinking. As seen in FIG. 6, the p65 target promoters are PCR amplified only in samples where p65-HT was incubated with HaloLink™ resin (FIG. 6, lanes 2, 4, 6). As a control, HaloLink™ alone was incubated with genomic HeLa DNA and treated with formaldehyde (lanes 1, 3, 5). The following p65 specific promoter regions were amplified using PCR: IKβα (300 bp) (lanes 1-2), IL-8 (182 bp) (lanes 3-4), and ICAM (165 bp) (lanes 5-6). The results indicate that in vitro protein:DNA crosslinking is specific after covalent attachment of a bait protein to a solid support.

### Example 9

### Preparation of Paramagnetic HaloTag® Particles

To encapsulate iron oxide, 0.375 g of iron (II, III) oxide was suspended in 25 mL of water and the mixture was sonicated for 10-15 minutes to disperse the iron oxide. 1.5 g of agarose (4 to 6% w/v) was added to the iron oxide, and the suspension was heated to reflux in a 3-neck (angled) 300 mL round bottom with an overhead stirrer. Separately, mineral oil (150 g)+Span 80 (sorbitan menoleate; 1.5 g) was heated to 90-100° C. When both solutions reached their respective target times, the oil/Span 80 solution was added to the iron oxide/agarose suspension all at once, and the stirrer speed was increased to create an emulsion. The emulsion was stirred for about 20 minutes at 90-100° C., and then the heat source was replaced with a water bath (about 15° C.). After 10-15 minutes, ice was added to the water bath to bring the final temperature to about 10° C. for 15 minutes. The mixture was transferred to a new beaker/flask and magnetized (settle). The oil was slowly poured off. The resin washed 4× with 250 mL acetone and then 2× with 250 mL of water.

Optionally, to cross-link agarose to the iron oxide, the drained agarose/iron oxide particles were mixed with one volume of 1.0 mol/L NaOH containing 10 g/L sodium borohydride and the suspension was stirred at 190 rpm in an incubator at 25° C. for 30 minutes. Epichlorohydrin was added to a final concentration of 2% (v/v) and the resulting reaction continued for 16-18 hours at room temperature. The cross-linked particles were then washed in deionized water thoroughly. The particles were then mixed with one volume of 2.0 mol/L NaOH containing 20 g/L sodium borohydride and incubated for 6 hours at 45° C. The particles were then washed thoroughly with deionized water until a neutral pH was reached.

To attach a ligand, e.g., a chloroalkane ligand, the drained agarose/iron oxide particles (e.g., 100 ml) were mixed with one volume of 0.6 mol/L NaOH. To this mixture sodium borohydride (150 mg) and 75 ml of 1,4-butanediol digylcidylether were added and the resin was kept in suspension for 16 to 24 hours. The resin was then washed thoroughly with 25% acetone followed by water. The epoxy-activated resin was suspended in 100 ml of 1 M ammonium hydroxide. This mixture was heated to 40° C. and kept in suspension for approximately 3 hours. The resin was washed thoroughly with water, followed by 25, 50, 75 and 100% acetone, and then with 100% dimethylformamide (DMF). The resin (125 to 150 mL) was suspended in DMF (about 200 mL) and 1.8 mmoles of PBI 400-10 (2-(2-(2-((4-nitrophenoxy)carbonyloxy)ethoxy)ethoxy)ethyl 2-(2-(6-chlorohexyloxy)ethoxy)ethylcarbamate) was added followed by 1 mL of triethylamine. This mixture was kept in suspension for 16 to 24 hours. The resin washed with DMF. The washed resin was again suspended in DMF (about 200 mL) and 1.5 mL of acetic anhydride was added followed by 1 mL of triethylamine, and this mixture was kept in suspension for 4 hours. The resin washed extensively and stored with 25% ethanol.

### REFERENCES

1. Chen, J. et. al. (1995) Identification of an 11-kDa FKBP12-rapamycin-binding domain within the 289-kDa FKBP12-rapamyscin-associated protein and characterization of a critical serine residue. PNAS192, 4947-51
2. Chinenoy, Y. and Kerppola T. K. (2001) Close encounters of many kinds: For-Jun interactions that mediate transcription regulatory specificity. Oncogene 19 2438-52.
3. Gerritsen, M. E. et. al (1997) CREB-binding protein/p300 are transcriptional coactivators of p65. Biochemistry 94 2927-2932.
4. Wells, J. and Farnham, P. J. (2002) Characterizing transcription factor binding sites using formaldehyde crosslinking and immunprecipitation. Methods 26, 48-56.
5. Martone, R., et. al (2003) Distribution of NF-KB-binding sites across human chromosome 22. PNAS 100, 12247-12252.
6. Nowak, D. E., Tian, B., and Brasier, A. R. (2005) Two-step cross-linking method for identification of NF-KB gene network by chromatin immunoprecipitation. Biotechniques 39, 715-728.
7. Kurdistani, S. K. and Grunstein M. (2003) In vivo protein-protein and protein-DNA crosslinking for genomewide binding microarray. Methods 31, 90-54

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method for capturing from a sample a target biomolecule that forms a complex with an interacting partner, comprising:
providing a support matrix having at least one ligand covalently coupled thereto, the at least one ligand capable of selective covalent attachment to a ligand-corresponding protein;
providing a capture complex formed by contacting a sample suspected of having a target biomolecule, an interacting partner for the target biomolecule and the ligand-corresponding protein;
treating the capture complex with a covalent cross-linking agent to form a covalently cross-linked capture complex; and
contacting the covalently cross-linked capture complex and the support matrix having the at least one ligand under conditions that permit the covalent attachment of the covalently cross-linked capture complex to the at least one ligand.

2. The method of claim 1 wherein the interacting partner is a protein.

3. The method of claim 2 wherein the target biomolecule is a protein, a nucleic acid, a lipid, or a carbohydrate.

4. The method of claim 1 wherein the support matrix comprises agarose.

5. The method of claim 1 wherein the at least one ligand is an alkylhalide.

6. The method of claim 5 wherein the alkylhalide is a chloroalkane.

7. The method of claim 1 wherein the ligand and the ligand-corresponding protein covalently attach through an ester or a thioether bond.

8. The method of claim 1 wherein the cross-linking agent is a reversible cross-linking agent.

9. The method of claim 1 wherein the ligand is an alkylated purine or an alkylated pyrimidine.

10. The method of claim 1 wherein the interacting partner and the ligand-corresponding protein form a fusion protein.

11. The method of claim 10 wherein the fusion protein is expressed from a nucleic acid sequence encoding the interacting partner and the ligand-corresponding protein in a single open reading frame.

12. A method for capturing from a sample a target biomolecule that forms a complex with an interacting biomolecule, comprising:
providing a support matrix having at least one ligand covalently coupled thereto, the at least one ligand capable of selective covalent attachment to a ligand-corresponding protein;
providing a capture complex formed by contacting a sample suspected of having a target biomolecule, an interacting biomolecule for the target biomolecule and the ligand-corresponding protein;
combining the capture complex and the support matrix under conditions that permit the covalent attachment of the capture complex to the at least one ligand; and
treating the combined capture complex and support matrix with a covalent cross-linking agent, thereby forming a covalently cross-linked capture complex attached to the support matrix.

13. The method of claim 12 wherein the interacting biomolecule is a protein.

14. The method of claim 13 wherein the target biomolecule is a protein, a nucleic acid, a lipid, or a carbohydrate.

15. The method of claim 12 wherein the support matrix comprises agarose.

16. The method of claim 12 wherein the ligand is an alkylhalide.

17. The method of claim 16 wherein the alkylhalide is a chloroalkane.

18. The method of claim 12 wherein the ligand and the ligand-corresponding protein covalently attach through an ester or a thioether bond.

19. The method of claim 12 wherein the cross-linking agent is a reversible cross-linking agent.

20. The method of claim 12 wherein the ligand is an alkylated purine or an alkylated pyrimidine.

21. The method of claim 12 wherein the interacting partner and the ligand-corresponding protein form a fusion protein.

22. The method of claim 21 wherein the fusion protein is expressed from a nucleic acid sequence encoding the interacting partner and the ligand-corresponding protein in a single open reading frame.

23. A method for capturing a target biomolecule that forms a complex with an interacting partner, comprising:
providing a support matrix having at least one ligand covalently coupled thereto, the at least one ligand capable of selective covalent attachment to a ligand-corresponding protein;
forming a capture complex having the support matrix, a target biomolecule, an interacting partner for the target biomolecule and the ligand-corresponding protein, wherein the ligand-corresponding protein is covalently attached to the at least one ligand coupled to the support matrix; and
treating the capture complex with a covalent cross-linking agent to form a covalently cross-linked capture complex.

24. The method of claim 23 wherein the interacting partner is a protein.

25. The method of claim 24 wherein the target biomolecule is a protein, a nucleic acid, a lipid, or a carbohydrate.

26. The method of claim 23 wherein the support matrix comprises agarose.

27. The method of claim 23 wherein the ligand is an alkylhalide.

28. The method of claim 27 wherein the alkylhalide is a chloroalkane.

29. The method of claim 23 wherein the ligand and the ligand-corresponding protein covalently attach through an ester or a thioether bond.

30. The method of claim 23 wherein the cross-linking agent is a reversible cross-linking agent.

31. The method of claim 23 wherein the ligand is an alkylated purine or an alkylated pyrimidine.

32. The method of claim 23 wherein the interacting partner and the ligand-corresponding protein form a fusion protein.

33. The method of claim 32 wherein the fusion protein is expressed from a nucleic acid sequence encoding the interacting partner and the ligand corresponding protein in a single reading frame.

34. A method for capturing a protein-protein interaction complex, comprising:
providing a support matrix having at least one ligand covalently coupled thereto, the at least one ligand capable of selective covalent attachment to a ligand-corresponding protein;
providing a capture complex having a set of interacting proteins and the ligand-corresponding protein;
treating the capture complex with a reversible cross-linking agent to form a covalently cross-linked capture complex, wherein the set of interacting proteins is covalently cross-linked; and
contacting the covalently cross-linked capture complex and the support matrix under conditions that permit the covalent attachment of the covalently cross-linked capture complex to the at least one ligand through the ligand-corresponding protein.

35. The method of claim 34 further comprising washing the support matrix having the covalently cross-linked capture complex.

36. The method of claim 35 further comprising subjecting the washed support matrix to conditions that reverse the covalent cross-linking of the set of interacting proteins, thereby allowing the release of at least one member of the set of interacting proteins.

37. A method for capturing the interaction of a polypeptide with a specific nucleic acid sequence, comprising:
providing a support matrix having at least one ligand covalently coupled thereto, the at least one ligand capable of selective covalent attachment to a ligand-corresponding protein;
providing a composition having a polypeptide that interacts with a specific nucleic acid sequence and the ligand-corresponding protein;
combining the composition and a sample suspected of having the specific nucleic acid sequence for a period of time and under conditions suitable for the polypeptide to bind to the nucleic acid sequence, thereby forming a mixture;
treating the mixture with a reversible cross-linking agent to form a covalently cross-linked complex having the polypeptide and the nucleic acid sequence; and
contacting the covalently cross-linked complex with the support matrix under conditions that permit the covalent capture of the covalently cross-linked capture complex to the at least one ligand through the ligand-corresponding protein.

38. The method of claim 37 further comprising washing the support matrix having the captured complex.

39. The method of claim 38 further comprising subjecting the washed support matrix to conditions that reverse the covalent cross-linking of the polypeptide and nucleic acid sequence.

40. The method of claim 38 or 39 further comprising subjecting the washed support matrix to a proteinase.

41. The method of claim 38, 39 or 40 further comprising amplifying the nucleic acid.

42. A method to isolate a complex, comprising:
a) providing a sample comprising one or more fusion proteins at least one of which comprises a mutant dehalogenase and a protein which may bind a molecule of interest, and a support matrix comprising paramagnetic agarose and one or more dehalogenase substrates, wherein the mutant dehalogenase comprises at least two amino acid substitutions relative to a corresponding wild-type dehalogenase, wherein the mutant dehalogenase forms a bond with the dehalogenase substrate, which bond is more stable than the bond formed between the corresponding wild-type dehalogenase and the substrate;
b) contacting the sample and the support matrix so as to form a mixture; and
c) isolating complexes formed between the one or more fusion proteins and the support matrix by subjecting the mixture to a magnetic field, which complexes are formed by the bond between the mutant dehalogenase in the fusion protein and the dehalogenase substrate.

43. The method of claim 42 wherein at least one amino acid substitution in the mutant dehalogenase is a substitution at an amino acid residue in the corresponding wild-type dehalogenase that is associated with activating a water molecule which cleaves the bond formed between the corresponding wild-type dehalogenase and the substrate or at an amino acid residue in the corresponding wild-type dehalogenase that forms an ester intermediate with the substrate, and wherein a second substitution is at an amino acid residue in the wild-type dehalogenase that is within the active site cavity and within 3 to 5 Å of a dehalogenase substrate bound to the wild-type dehalogenase, wherein at least one substitution is at a position corresponding to amino acid residue 106 or 272 of a Rhodococcus rhodochrous dehalogenase, and wherein the second substitution is at a position corresponding to amino acid residue 175, 176 or 273 of a Rhodococcus rhodochrous dehalogenase

44. The method of claim 43 wherein the substituted amino acid at the position corresponding to amino acid residue 272 is asparagine, phenylalanine, glycine or alanine.

45. The method of claim 43 wherein the substituted amino acid at the position corresponding to amino acid residue 175 is methionine, valine, glutamate, aspartate, alanine, leucine, serine or cysteine, wherein the substituted amino acid at the position corresponding to amino acid residue 176 is serine, glycine, asparagine, aspartate, threonine, alanine or arginine, or wherein the substituted amino acid at the position corresponding to amino acid residue 273 is leucine, methionine or cysteine.

46. The method of claim 42 wherein the support matrix comprises paramagnetic agarose-linker-A-X, wherein the linker is a branched or unbranched carbon chain comprising from 2 to 30 carbon atoms, which chain optionally includes one or more double or triple bonds, and which chain is optionally substituted with one or more hydroxy or oxo (═O) groups, wherein one or more of the carbon atoms in the chain is optionally replaced with a non-peroxide —O—, —S— or —NH—, wherein the linker-A separates the paramagnetic agarose and X by at least 11 atoms, wherein A is (CH₂)ₙ and n 2-10, wherein A-X is the substrate for the dehalogenase, and wherein X is a halogen.


SEQUENCE LISTING (12 sequences)

SEQ ID NO: 1 - DNA, 19 bp, Homo sapiens
gggccatcagttgcaaatc

SEQ ID NO: 2 - DNA, 20 bp, Homo sapiens
ttccttccggtggtttcttc

SEQ ID NO: 3 - DNA, 15 bp, Homo sapiens
ggttggcagtattta

SEQ ID NO: 4 - DNA, 15 bp, Homo sapiens
gcctcgctggccgct

SEQ ID NO: 5 - DNA, 20 bp, Homo sapiens
gacgaccccaattcaaatcg

SEQ ID NO: 6 - DNA, 19 bp, Homo sapiens
tcaggctcggggaatttcc

SEQ ID NO: 7 - DNA, 20 bp, Homo sapiens
tactagcggttttacgggcg

SEQ ID NO: 8 - DNA, 24 bp, Homo sapiens
tcgaacaggaggagcagagagcga

SEQ ID NO: 9 - DNA, 20 bp, Homo sapiens
atggttgccactggggatct

SEQ ID NO: 10 - DNA, 20 bp, Homo sapiens
tgccaaagcctaggggaaga

SEQ ID NO: 11 - DNA, 21 bp, Homo sapiens
gtcgtcaccatcacctgaaaa

SEQ ID NO: 12 - DNA, 20 bp, Homo sapiens
cagagtgtttccacctgcat